(19) United States
(12) Patent Application Publication
(10) Pub. No.: US 2023/0233154 A1
(43) Pub. Date: Jul. 27, 2023

(54) FEATURE EXTRACTION METHOD FOR PHOTOPLETHYSMOGRAPHY SIGNAL

(71) Applicant: National Central University, Taoyuan City (TW)

(72) Inventors: Pei-Yun Tsai, Taipei City (TW); Tzung-Dau Wang, Taipei City (TW); Hung-Ju Lin, Taipei City (TW)

(21) Appl. No.: 17/724,477

(22) Filed: Apr. 19, 2022

(30) Foreign Application Priority Data
Jan. 27, 2022 (TW) 111103696

(51) Int. Cl. A61B 5/00; A61B 5/0295

(52) U.S. Cl. CPC A61B 5/7239; A61B 5/0295

(57) ABSTRACT

A feature extraction method for photoplethysmography includes obtaining a photoplethysmography (PPG) signal; calculating a first-order derivative photoplethysmography (FDPPG) signal, a second-order derivative photoplethysmography (SDPPG) signal and a third-order derivative photoplethysmography (TDPPG) signal from the PPG signal, wherein the SDPPG signal has multiple feature points; and performing a feature extraction operation that exploits the property of the TDPPG signal to impute the missing feature points of the SDPPG signal. Moreover, the feature extraction operation further includes: resolving the ambiguous feature points of the SDPPG signal.

This application claims the benefit of Taiwan Patent Application Serial No. 111103696, filed Jan. 27, 2022, the subject matter of which is incorporated herein by reference.

### BACKGROUND OF INVENTION

### 1. Field of the Invention

The present invention relates to a feature extraction method. More particularly, the present invention relates to a feature extraction method for photoplethysmography.

### 2. Description of the Prior Art

With the advances in sensors and integrated circuits, wearable devices are prosperously developed in diverse scenarios, such as applications in ambient-assisted living, in sports training, and for diagnostic support. Among the wearable devices for pervasive health care enabling long-term health monitoring, non-invasive sensing of cardiovascular signals has become a trend in biomedical consumer products. Particularly, photoplethysmography (PPG), a low-cost optical device, can sense the blood volume changes from light intensity during the cardiac cycle.

Feature extraction from the PPG signal is an essential step to analyze vascular and hemodynamic information. By calculating the first-order derivative photoplethysmography (FDPPG) signal and the second-order derivative photoplethysmography (SDPPG) of the PPG signal, the feature points in these signals can be obtained, which is helpful for the subsequent model establishment to analyze the health status.

FIG. 1 is a photoplethysmography signal schematic diagram with standard morphology feature, wherein the upper half shows the PPG signal, and the lower half simultaneously shows the FDPPG signal and the SDPPG signal. Please refer to FIG. 1. In the standard morphology feature, the SDPPG has multiple clearly distinguishable feature points, such as feature point a, feature point b, feature point c, feature point d, and feature point e. By confirming the positions of these feature points, the PPG signal can be successfully decomposed into five Gaussian component waves which correspond to the possible pulsatile waves of the human body. By analyzing these signals, health indicators such as blood pressure, pulse wave velocity (PWV) and vascular age can be further estimated.

FIG. 2 is a photoplethysmography signal schematic diagram with non-standard morphology feature, wherein the left half shows the morphology feature of the ambiguous feature points of the SDPPG signal, and the right half shows the morphology feature of the missing feature points of the SDPPG signal. Please refer to FIG. 1 and FIG. 2 at the same time. Due to the different characteristics of each person's blood vessels, the prior feature extraction process sometimes results in missing or even ambiguous feature points when processing the SDPPG signal. This not only makes it difficult to decompose the PPG signal into the correct Gaussian component waves, but also causes a large deviation in estimating the values of health indicators.

### SUMMARY OF THE INVENTION

The present invention provides a feature extraction method for photoplethysmography, comprising: obtaining a photoplethysmography (PPG) signal; calculating a first-order derivative photoplethysmography (FDPPG) signal, a second-order derivative photoplethysmography (SDPPG) signal and a third-order derivative photoplethysmography (TDPPG) signal from the PPG signal, wherein the SDPPG signal has multiple feature points; and performing a feature extraction operation that exploits the property of the TDPPG signal to impute the missing feature points of the SDPPG signal.

According to one embodiment of the present invention, the feature extraction operation may further comprise: resolving the ambiguous feature points of the SDPPG signal. Besides, the feature extraction operation may also comprise: determining the regional extremum of the TDPPG signal to impute the missing feature points of the SDPPG signal.

According to one embodiment of the present invention, the feature extraction operation may comprise: calculating multiple zero-crossing points of the TDPPG signal; setting a search interval during one cardiac cycle duration of the PPG signal, searching a discrimination zero-crossing point from the zero-crossing points in the search interval, and taking the time of the discrimination zero-crossing point as the time of a feature point e; taking the time of an initial zero-crossing point from the zero-crossing points as the time of a feature point a, and taking the zero-crossing points of the TDPPG signal in the interval from the feature point a to the feature point e as multiple feature zero-crossing points; calculating multiple regional extreme points of the TDPPG signal in the interval from the feature point a to the feature point e, determining a first maximum point of the regional extreme points, taking the time of the zero-crossing point of the first ascending slope from the feature zero-crossing points as the time of a feature point b if the amplitude of the first maximum point is greater than zero, and taking the time of the first maximum point as the time of the feature point b and the time of a feature point c1 if the amplitude of the first maximum point is less than or equal to zero; determining a second minimum point of the regional extreme points, taking the time of the zero-crossing point of the first descending slope from the feature zero-crossing points as the time of the feature point c1 if the amplitude of the second minimum point is less than zero, and taking the time of the second minimum point as the time of the feature point c1 and the time of a feature point d1 if the amplitude of the second minimum point is greater than or equal to zero; and determining a second maximum point of the regional extreme points, and taking the time of the zero-crossing point of the second ascending slope from the feature zero-crossing points as the time of the feature point d1.

According to one embodiment of the present invention, the feature extraction operation may further comprise: determining the second maximum point of the regional extreme points, taking the time of the second maximum point as the time of the feature point d1 and the time of a feature point c2 and taking the time of the zero-crossing point of the third ascending slope from the feature zero-crossing points as the time of a feature point d2 if the amplitude of the second maximum point is less than or equal to zero; and determining whether the regional extreme points include a third minimum point, taking the feature point c1 and the feature point d1 as a feature point c and a feature point d respectively if the third minimum point does not exist, taking the time of the zero-crossing point of the second descending slope from the feature zero-crossing points as the time of the feature point c2 and taking the time of the zero-crossing point of the third ascending slope from the feature zero-crossing points as the time of the feature point d2 if the third minimum point exists and the amplitude of the third minimum point is less than zero, and taking the time of the third minimum point as the time of the feature point c2 and the time of the feature point d2 if the third minimum point exists and the amplitude of the third minimum point is greater than or equal to zero.

According to one embodiment of the present invention, the feature extraction operation may further comprise: taking the feature point c2 and the feature point d2 as the feature point c and the feature point d respectively if the feature point c2 and the feature point d2 exist.

According to one embodiment of the present invention, the initial zero-crossing point is the zero-crossing point of the first descending slope from the zero-crossing points of the TDPPG signal.

According to one embodiment of the present invention, the feature extraction method for photoplethysmography may further comprise: performing the feature extraction operation that exploits the property of the SDPPG signal to impute a maximum slope feature point of the FDPPG signal. In particular, the feature extraction operation comprises: determining the regional extremum of the SDPPG signal to impute the maximum slope feature point of the FDPPG signal.

According to one embodiment of the present invention, the feature extraction operation may comprise: marking a feature point a of the SDPPG signal; calculating a first regional minimum point of the SDPPG signal behind the feature point a; and determining the first regional minimum point, taking the time of the zero-crossing point the first descending slope of the SDPPG signal as the time of the maximum slope feature point if the amplitude of the first regional minimum point is less than zero, and taking the time of the first regional minimum point as the time of the maximum slope feature point if the amplitude of the first regional minimum point is greater than or equal to zero.

This Summary is provided to introduce a selection of concepts in a simplified form and further detailed description are provided below. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is a photoplethysmography signal schematic diagram with standard morphology features;

FIG. 2 is a photoplethysmography signal schematic diagram with non-standard morphology features;

FIG. 3 is a schematic diagram illustrating the variation of the SDPPG signal corresponding to the TDPPG signal;

FIG. 4 is a flowchart of the feature extraction method for photoplethysmography according to one embodiment of the present invention;

FIG. 5 is a flowchart of the feature extraction operation according to one embodiment of the present invention; and

FIG. 6 is a flowchart of the feature extraction operation according to another embodiment of the present invention.

### DESCRIPTION OF THE PREFERRED EMBODIMENT

Generally speaking, the feature points of the SDPPG signal are defined by the multiple regional extreme points of the SDPPG signal. In the case of missing or ambiguous feature points, it is difficult to directly determine the feature points of the SDPPG signal, and it is necessary to use the third-order derivative photoplethysmography (TDPPG) signal to determine Specifically, the zero-crossing points of the TDPPG signal correspond to the regional extreme points of the SDPPG signal. When the SDDPG signal has non-standard morphology feature, the zero-crossing point of the TDPPG signal will change accordingly.

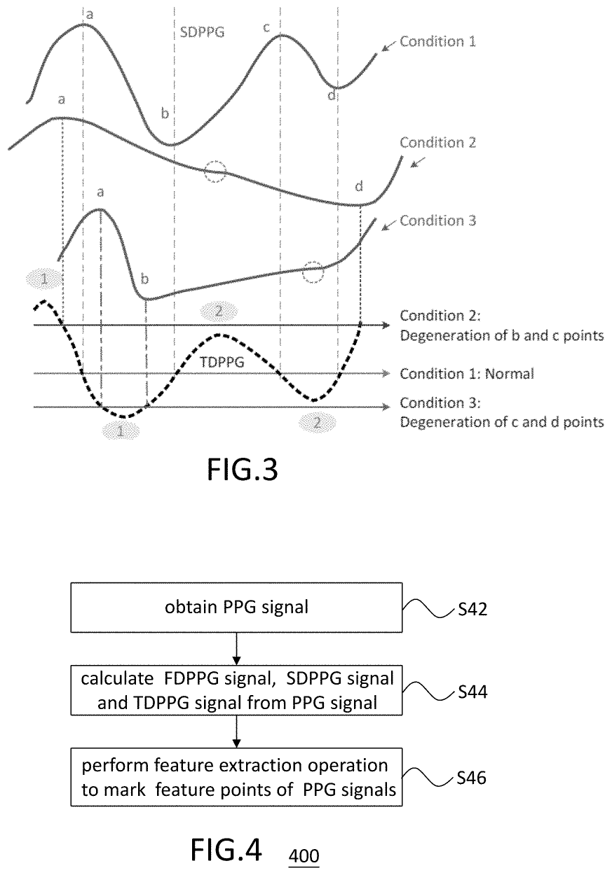

FIG. 3 is a schematic diagram illustrating the variation of the SDPPG signal corresponding to the TDPPG signal, wherein the upper half shows the SDPPG signals with different morphology features, and the lower half shows the corresponding TDPPG signal. Please refer to FIG. 3. Condition 1 is a standard morphology feature, SDDPG signal has clearly distinguishable feature point a, feature point b, feature point c, and feature point d, and the corresponding TDPPG signal in the observed region will cross the X-axis four times to generate four zero-crossing points associating the feature point a, the feature point b, the feature point c, and the feature point d, respectively.

However, in condition 2 of degeneration of the feature point b and the feature point c, the second regional maximum point of the TDPPG signal is lower than the X-axis, thus the TDPPG signal in the interested region will only cross the X-axis twice to generate two zero-crossing points associating the feature point a and the feature point d respectively. Similarly, in condition 3 of degeneration of the feature point c and the feature point d, the second regional minimum point of the TDPPG signal is higher than the X-axis, thus the TDPPG signal in the interested region will only cross the X-axis twice to generate two zero-crossing points associating the feature point a and the feature point b respectively.

In the present invention, the amplitudes of the regional extreme points of the TDPPG signal relative to the X-axis are used to determine the degeneration of the feature points and the positions of the regional extreme points are used to impute the missing feature points. Please refer to FIG. 3 again. In condition 2, when the amplitude of the second regional maximum point of the TDPPG signal is less than or equal to zero, the second regional maximum point of the TDPPG corresponds to the imputed feature point of the SDPPG signal. That is, the time of the second regional maximum point of the TDPPG signal is taken as the time of the missing feature points b and c of the SDPPG signal to impute the feature points b and c.

Similarly, in condition 3, when the amplitude of the second regional minimum point of the TDPPG signal is greater than or equal to zero, the second regional minimum point of the TDPPG corresponds to the imputed feature point of the SDPPG signal. That is, the time of the second regional minimum point of the TDPPG signal is taken as the time of the missing feature points c and d of the SDPPG signal to impute the feature points c and d.

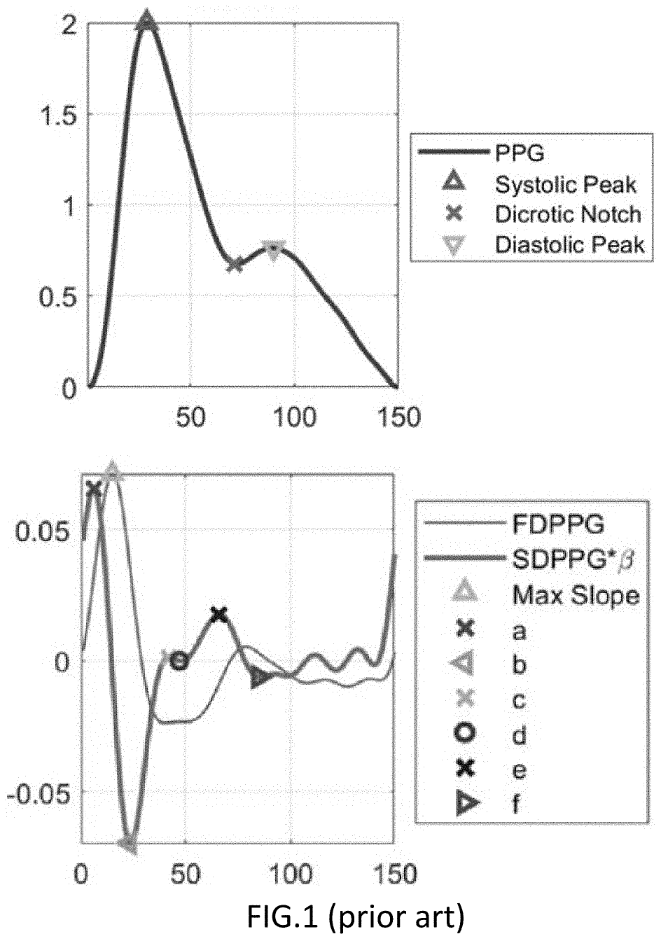

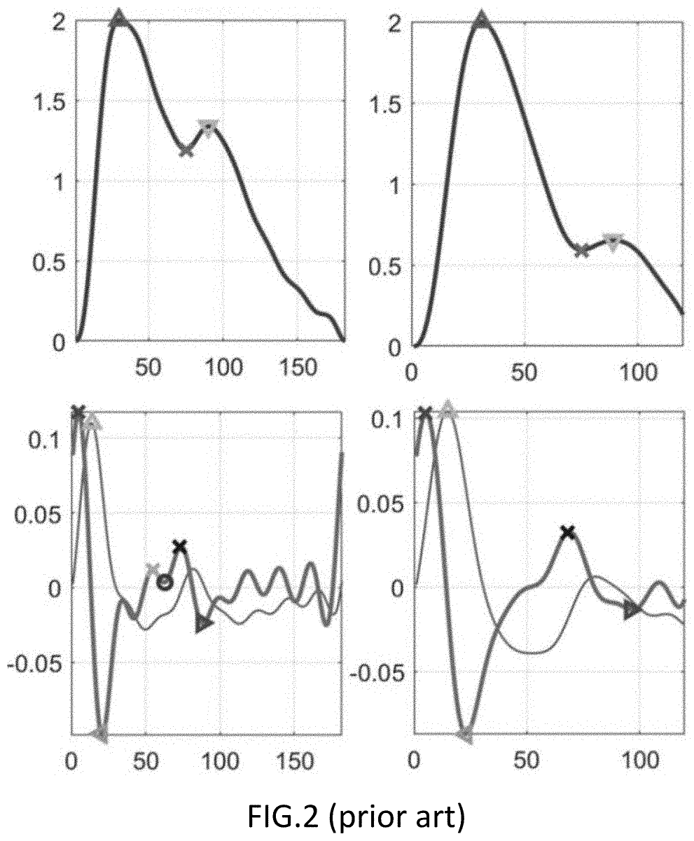

It should be noted that in the previous two paragraphs, in order to facilitate the understanding of the concept of the present invention with the schematic diagrams, the ordinal numbers of the regional extreme points of the TDPPG signal are calculated based on the interval range shown in FIG. 3, rather than the cardiac cycle duration shown in FIG. 1. Those skilled in the art can understand that the present invention uses the regional extreme points of the TDPPG signal to determine whether the morphology feature points are missing or not, and the ordinal numbers of the regional extreme points will vary according to the selected interval.

Besides, the case of the ambiguous feature points refers to the occurrence of multiple sets of feature points c and d of morphology feature, and the corresponding TDPPG signal has unexpected regional extreme points. The present invention first divides these ambiguous feature points into the feature points c1, d1 and the feature points c2, d2, and finally decides to take the feature points c1, d1 or the feature points c2, d2 as the feature points c, d.

Based on the gist of the foregoing description, the feature extraction method of the present invention will be specifically described below. It should be noted that, without departing from the spirit and scope of the present invention, any slight changes or sequence changes of any steps still fall within the protection scope of the present invention.

FIG. 4 is a flowchart of the feature extraction method for photoplethysmography according to one embodiment of the present invention. Please refer to FIG. 4. The feature extraction method for photoplethysmography 400 of the present invention comprises the following steps. The first step is to obtain the photoplethysmography (PPG) signal as shown in step S42. In this embodiment, for example, a wrist-wearable or finger-wearable sensing device is used to measure the PPG signal, but the present invention does not limit the type of sensing device. For instance, an earlobe-wearable sensing device can also be used.

The next step is to calculate the FDPPG signal, the SDPPG signal and the TDPPG signal from the PPG signal as shown in step S44. Please also refer to FIG. 1 at the same time. Those PPG signals have multiple feature points to be marked, so as to facilitate subsequent estimation of blood pressure, PWV and other health indicators. For example, the PPG signal has the systolic peak feature point, the diastolic peak feature point and the dicrotic notch feature point, while the FDPPG signal has the maximum slope feature point, and the SDPPG signal has the feature point a, the feature point b, the feature point c, the feature point d, the feature point e and the feature point f.

The next step is to perform the feature extraction operation to mark the feature points of those PPG signals as shown in step S46. In one embodiment, if some feature points of the SDPPG signal are missing, the missing feature points of the SDPPG signal can be imputed by performing the feature extraction operation that exploits the property of the TDPPG signal. In another embodiment, if some feature points of the SDPPG signal are ambiguous, the ambiguous feature points of the SDPPG signal can be resolved by performing the feature extraction operation that exploits the property of the TDPPG signal. In yet another embodiment, if the maximum slope feature point of the FDPPG signal is missing, the missing maximum slope feature point of the FDPPG signal can be imputed by performing the feature extraction operation that exploits the property of the SDPPG signal.

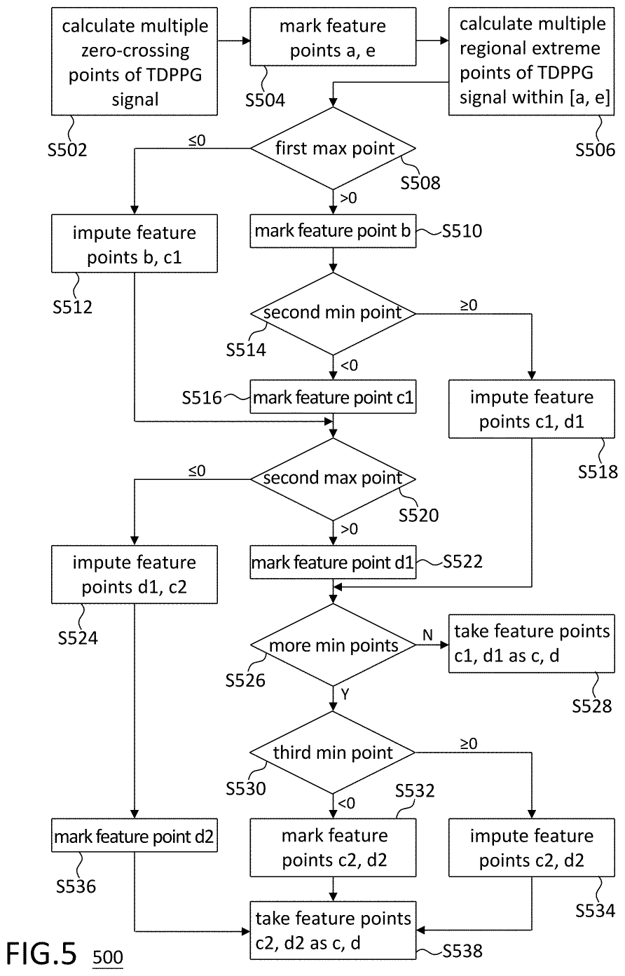

FIG. 5 is a flowchart of the feature extraction operation according to one embodiment of the present invention, so as to impute the missing feature points of the SDPPG signal, and simultaneously resolve the ambiguous feature points of the SDPPG signal. Please refer to FIG. 5. The feature extraction operation 500 of this embodiment comprises the following steps. The first step is to calculate multiple zero-crossing points of the TDPPG signal as shown in step S502, and these zero-crossing points can be further classified into the zero-crossing points of the ascending slope and the zero-crossing points of the descending slope to indicate whether the TDPPG signal crosses the X-axis from bottom to top or crosses the X-axis from top to bottom, respectively.

The next step is to mark the feature point a and the feature point e of the SDDPG signal as shown in step S504. Specifically, the feature point a and the feature point e are the regional extreme points of the SDPPG signal, and correspond to specific zero-crossing points of the TDPPG signal respectively.

The feature point e is usually located near the junction of systole and diastole, so a search interval can be set within one cardiac cycle duration of the PPG signal, and the zero-crossing point of the TDPPG signal in this search interval can be used as the discrimination zero-crossing point, and then the time of the discrimination zero-crossing point is taken as the time of the feature point e to mark the feature point e. The search interval is, for example, set to [α₁+β₁NTs, α₂+β₂NTs], where NTs represents the cardiac cycle duration, and α₁, β₁, α₂ and β₂ are parameter values. In this embodiment, the values of α₁, β₁, α₂ and β₂ are respectively set to 0.16, 0.1, 0.3 and 0.1, which are sufficient to cover most situations where the heart rate is lower than 120 bpm during stationary activities. It should be noted that the present invention does not limit the setting formula or parameter value of the search interval, and those skilled in the art can fine-tune the relevant parameters and still fall within the scope of the present invention.

The feature point a is the first feature point within the cardiac cycle duration, so the zero-crossing point of the first descending slope from the zero-crossing points can be used as the initial zero-crossing point, and then the time of the initial zero-crossing point is taken as the time of the feature point a to mark the feature point a.

After completing the marking of the feature point a and the feature point e, the next step is to mark the feature points b, c, d in the interval from the feature point a to the feature point e of the SDPPG, especially imputation or resolution can be performed when missing or ambiguous morphology feature occurs for identifying the feature points b, c, d. In order to focus on the interval from the feature point a to the feature point e and to avoid confusion, the present invention further redefines these zero-crossing points in the interval from the feature point a to the feature point e as multiple feature zero-crossing points.

Please refer to FIG. 5 again. The next step is to calculate multiple regional extreme points of the TDPPG signal in the interval from the feature point a to the feature point e as shown in step S506. By determining these regional extreme points, it is possible to confirm whether missing or ambiguous morphology feature occurs for identifying the feature points, and then perform imputation or resolution.

The next step is to determine the first maximum point of these regional extreme points as shown in step S508. If the amplitude of the first maximum point is greater than zero, it means that missing morphology feature does not occur for identifying the feature point b. Then, the time of the zero-crossing point of the first ascending slope from these feature zero-crossing points is taken as the time of the feature point b to mark the feature point b as shown in step S510.

Conversely, if the amplitude of the first maximum point is less than or equal to zero, it means that missing morphology feature occurs for identifying feature points b, c1. Then, the time of the first maximum point is taken as the time of the feature points b, c1 to impute the feature points b, c1 as shown in step S512. It is worth noting that this embodiment adopts the process of determining the feature points in sequence, and in the current step, it has not yet been confirmed whether ambiguous morphology feature occurs for identifying the feature points c, d or not. Therefore, the feature point c1 is temporarily marked to wait for the subsequent process to decide whether to take the feature point c1 as the feature point c. Similarly, marking the feature points d1, c2, d2 are also of the same concept.

After the step S510 of marking the feature point b, the next step is to determine the second minimum point of these regional extreme points as shown in step S514. If the amplitude of the second minimum point is less than zero, it means that missing morphology feature does not occur for identifying the feature point c1. Then, the time of the zero-crossing point of the first descending slope from these feature zero-crossing points is taken as the time of the feature point c1 to mark the feature point c1 as shown in step S516.

Conversely, if the amplitude of the second minimum point is greater than or equal to zero, it means that missing morphology feature occurs for identifying the feature points c1, d1. Then, the time of the second minimum point is taken as the time of the feature points c1, d1 to impute the feature points c1, d1 as shown in step S518.

After the step S516 of marking the feature point c1, the next step is to determine the second maximum point of these regional extreme points as shown in step S520. Incidentally, after the aforementioned step S512 of imputing the feature points b, c1 with missing morphology feature, the continuation process is also step S520.

If the amplitude of the second maximum point is greater than zero, it means that missing morphology feature does not occur for identifying the feature point d1. Then, the time of the zero-crossing point of the second ascending slope from these feature zero-crossing points is taken as the time of the feature point d1 to mark the feature point d1 as shown in step S522.

Conversely, if the amplitude of the second maximum point is less than or equal to zero, it means that missing morphology feature occurs for identifying the feature points d1, c2. Then, the time of the second maximum point is taken as the time of the feature points d1, c2 to impute the feature points d1, c2 as shown in step S524. It is worth mentioning that when proceeding to step S522 or step S524, the imputation process related with missing feature points c1, d1 has been completed, and then the resolution process of ambiguous feature points will be performed.

After the step S522 of marking the feature point d1, the next step is to determine whether the regional extreme points include more minimum points as shown in step S526, which is to determine whether the regional extreme points include the third minimum point. Incidentally, after the aforementioned step S518 of imputing the feature points c1, d1 given missing morphology feature, the continuation process is also step S526.

If the third minimum point does not exist, it means that ambiguous morphology feature does not occur for identifying the feature points c, d. Then, the feature points c1, d1 are respectively taken as the feature points c, d as shown in step S528. Conversely, if the third minimum point exists, it means that ambiguous morphology feature occurs for identifying the feature points c, d, and then the next step is to determine the third minimum point as shown in step S530.

If the amplitude of the third minimum point is less than zero, it means that missing morphology feature does not occur for identifying the feature points c2, d2. Then, the time of the zero-crossing point of the second descending slope from these feature zero-crossing points is taken as the time of the feature point c2 to mark the feature point c2, and the time of the zero-crossing point of the third ascending slope from these feature zero-crossing points is taken as the time of the feature point d2 to mark the feature point d2, as shown in step S532.

Conversely, if the amplitude of the third minimum point is greater than or equal to zero, it means that missing morphology feature occurs for identifying the feature points c2, d2. Then, the time of the third minimum point is taken as the time of the feature points c2, d2 to impute the feature point c2, d2 as shown in step S534.

Incidentally, after the aforementioned step S524 of imputing the feature points d1, c2, it also indicates that ambiguous morphology feature occurs for identifying the feature points c, d. Then, the zero-crossing of the third ascending slope from these feature zero-crossing points is taken as the time of the feature point d2 to mark the feature point d2 as shown in step S536.

It is worth noting that, the feature extraction operation 500 of this embodiment integrates imputation of missing feature points of the SDPPG signal and resolution of ambiguous feature points of the SDPPG signal into one single process, but the present invention does not limit the process form. Those skilled in the art can only use the imputation process of missing feature points or the resolution process of ambiguous feature points according to requirements, which still falls within the scope of the present invention.

In addition, the ordinal numbers of the ascending slope or the descending slope to identify the zero-crossing point mentioned above are defined by the TDPPG signal of common morphology features in the interested region. Those skilled in the art can easily understand that if non-standard morphology feature occurs at a feature point, the ordinal numbers of the ascending slope or the descending slope may be changed and correspondingly adjusted, which will not be repeated here.

Please refer to FIG. 5 again. After the step S532 of marking the feature points c2, d2, the step S534 of imputing the feature points c2, d2, and the step S536 of marking the feature point d2, the next step is to take the feature points c2, d2 as the feature points c, d as shown in step S538. In this embodiment, if the feature points c2, d2 exist, the feature points c, d are preferentially set as the feature points c2, d2 instead of the feature points c1, d1. However, the present invention does not limit the selection method of the feature points c, d when ambiguous morphology feature occurs for identifying the feature points c, d. For example, in other embodiments of the present invention, the feature points c, d can also be set by considering the relative positions of the feature points c1, d1 and the feature points c2, d2 in the interval from the feature point a to the feature point e.

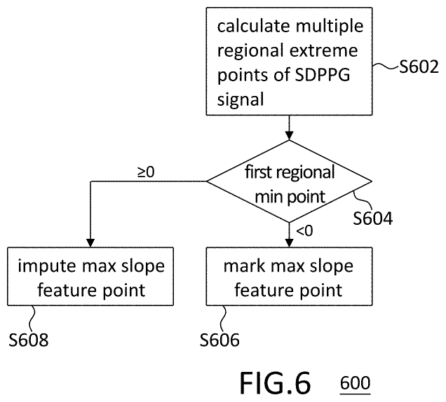

FIG. 6 is a flowchart of the feature extraction operation according to another embodiment of the present invention to impute the missing maximum slope feature point of the FDPPG signal. Please refer to FIG. 6. The feature extraction operation 600 of this embodiment comprises the following steps. The first step is to calculate multiple regional extreme points of the SDPPG signal as shown in step S602, specifically, to calculate the first regional minimum point of the SDPPG signal. By determining the first regional minimum point as shown in step S604, it can be confirmed whether missing morphology feature occurs for identifying the maximum slope feature point, and then imputation is performed.

It should be noted that this embodiment still focuses on the signal changes behind the feature point a, so the aforementioned first regional minimum point of the SDPPG signal is performed ordinal calculation after the feature point a, not the interval of the cardiac cycle duration. In addition, the marking process of the feature point a has been described above, and will not be repeated here.

If the amplitude of the first regional minimum point is less than zero, the time of the zero-crossing point of the first descending slope of the SDPPG signal is taken as the time of the maximum slope feature point to mark the maximum slope feature point as shown in step S606. Conversely, if the amplitude of the first regional minimum point is greater than or equal to zero, the time of the first regional minimum point is taken as the time of the maximum slope feature point to impute the maximum slope feature point as shown in step S608.

In view of the foregoing, the present invention can confirm whether missing or ambiguous morphological feature occurs for identifying the feature points of low-order derivative PPG signal from the amplitude of the regional extreme point of high-order derivative PPG signal, and then perform imputation or resolution. Furthermore, the present invention can perform imputation and resolution on the SDPPG signal with simultaneous occurrence of missing and ambiguous morphology features through systematic procedure steps, thereby effectively improving processing efficiency.

Besides, the feature extraction method of the present invention can effectively impute the missing feature points or resolve the ambiguous feature points, and according to experimental data, the success rate can be greatly increased to more than 98.7%. Therefore, the feature extraction method of the present invention can effectively improve the subsequent estimation of blood pressure, PWV, vascular age and other health indicators, and is helpful for the development of smart health wearable devices.

Although the present invention has been described with reference to the above embodiments, these embodiments are not intended to limit the present invention. It will be apparent to those skilled in the art that various modifications and variations can be made without departing from the scope or spirit of the present invention. Therefore, the scope of the present invention shall be defined by the appended claims.

What is claimed is:

1. A feature extraction method for photoplethysmography, comprising:
obtaining a photoplethysmography (PPG) signal;
calculating a first-order derivative photoplethysmography (FDPPG) signal, a second-order derivative photoplethysmography (SDPPG) signal and a third-order derivative photoplethysmography (TDPPG) signal from the PPG signal, wherein the SDPPG signal has multiple feature points; and
performing a feature extraction operation that exploits the property of the TDPPG signal to impute the missing feature points of the SDPPG signal.

2. The feature extraction method for photoplethysmography of claim 1, wherein the feature extraction operation further comprises: resolving the ambiguous feature points of the SDPPG signal.

3. The feature extraction method for photoplethysmography of claim 1, wherein the feature extraction operation comprises: determining the regional extremum of the TDPPG signal to impute the missing feature points of the SDPPG signal.

4. The feature extraction method for photoplethysmography of claim 3, wherein the feature extraction operation comprises:
calculating multiple zero-crossing points of the TDPPG signal;
setting a search interval during one cardiac cycle duration of the PPG signal, searching a discrimination zero-crossing point from the zero-crossing points of the TDPPG signal in the search interval, and taking the time of the discrimination zero-crossing point as the time of a feature point e;
taking the time of an initial zero-crossing point from the zero-crossing points of the TDPPG signal as the time of a feature point a, and taking the zero-crossing points of the TDPPG signal in the interval from the feature point a to the feature point e as multiple feature zero-crossing points;
calculating multiple regional extreme point of the TDPPG signal in the interval from the feature point a to the feature point e, determining a first maximum point of the regional extreme points, taking the time of the zero-crossing point of the first ascending slope from the feature zero-crossing points as the time of a feature point b if the amplitude of the first maximum point is greater than zero, and taking the time of the first maximum point as the time of the feature point b and the time of a feature point c1 if the amplitude of the first maximum point is less than or equal to zero;
determining a second minimum point of the regional extreme points of the TDPPG signal, taking the time of the zero-crossing point of the first descending slope from the feature zero-crossing points as the time of the feature point c1 if the amplitude of the second minimum point is less than zero, and taking the time of the second minimum point as the time of the feature point c1 and the time of a feature point d1 if the amplitude of the second minimum point is greater than or equal to zero; and
determining a second maximum point of the regional extreme points of the TDPPG signal, and taking the time of zero-crossing point of the second ascending slope from the feature zero-crossing points as the time of the feature point d1.

5. The feature extraction method for photoplethysmography of claim 4, wherein the feature extraction operation further comprises:
determining the second maximum point of the regional extreme points of the TDPPG signal, taking the time of the second maximum point as the time of the feature point d1 and the time of a feature point c2 and taking the time of the zero-crossing point of the third ascending slope from the feature zero-crossing points as the time of a feature point d2 if the amplitude of the second maximum point is less than or equal to zero; and
determining whether the regional extreme points of the TDPPG signal include a third minimum point, taking the feature point c1 and the feature point d1 as a feature point c and a feature point d respectively if the third minimum point does not exist, taking the time of the zero-crossing point of the second descending slope from the feature zero-crossing points as the time of the feature point c2 and taking the time of the zero-crossing point of the third ascending slope from the feature zero-crossing points as the time of the feature point d2 if the third minimum point exists and the amplitude of the third minimum point is less than zero, and taking the time of the third minimum point as the time of the feature point c2 and the time of the feature point d2 if the third minimum point exists and the amplitude of the third minimum point is greater than or equal to zero.

6. The feature extraction method for photoplethysmography of claim 5, wherein the feature extraction operation further comprises: taking the feature point c2 and the feature point d2 as the feature point c and the feature point d respectively if the feature point c2 and the feature point d2 exist.

7. The feature extraction method for photoplethysmography of claim 4, wherein the initial zero-crossing point of the TDPPG signal is the zero-crossing point of the first descending slope from the zero-crossing points.

8. The feature extraction method for photoplethysmography of claim 1, further comprising: performing the feature extraction operation that exploits the property of the SDPPG signal to impute a maximum slope feature point of the FDPPG signal.

9. The feature extraction method for photoplethysmography of claim 8, wherein the feature extraction operation comprises: determining the regional extremum of the SDPPG signal to impute the maximum slope feature point of the FDPPG signal.

10. The feature extraction method for photoplethysmography of claim 9, wherein the feature extraction operation comprises:
marking a feature point a of the SDPPG signal;
calculating a first regional minimum point of the SDPPG signal behind the feature point a; and
determining the first regional minimum point, taking the time of the zero-crossing point of the first descending slope of the SDPPG signal as the time of the maximum slope feature point if the amplitude of the first regional minimum point is less than zero, and taking the time of the first regional minimum point as the time of the maximum slope feature point if the amplitude of the first regional minimum point is greater than or equal to zero.
